(19) United States
(12) Patent Application Publication
(10) Pub. No.: US 2024/0153599 A1
(43) Pub. Date: May 9, 2024

(54) SYSTEM AND METHOD FOR AUTOMATING SELF-EXPERIMENTATION BASED ON LIFELOG DATA FOR HEALTH BEHAVIOR CHANGE

(71) Applicant: Korea Advanced Institute Of Science And Technology, Daejeon (KR)

(72) Inventors: Uichin LEE, Daejeon (KR); Gyuwon JUNG, Daejeon (KR); Heepyung KIM, Daejeon (KR); Sangjun PARK, Daejeon (KR)

(21) Appl. No.: 18/190,784

(22) Filed: Mar. 27, 2023

(30) Foreign Application Priority Data
Nov. 9, 2022 (KR) 10-2022-0148907
Jan. 17, 2023 (KR) 10-2023-0006560

(51) Int. Cl. G16H 10/20

(52) U.S. Cl. CPC G16H 10/20

(57) ABSTRACT

Disclosed is a system and method for supporting automated self-experimentation based on lifelog data for health behavior change. The system includes a data collection unit configured to collect data related to a user from a plurality of smart terminals and preprocess the data as variables that represent a lifelog of the user; a causal inference unit configured to establish a causal relationship hypothesis between the variables of the user and infer a causal relationship between the variables collected from the user using a statistical analysis method; a variable recommendation unit configured to recommend a variable for verifying the causal relationship through self-experimentation for the user based on an inference result acquired from the causal inference unit and an interaction of the user with the system; and a self-experimentation planning unit configured to plan and conduct an experiment to verify the causal relationship through the self-experimentation for the user.

### CROSS-REFERENCE TO RELATED APPLICATION

This application claims the priority benefit of Korean Patent Application No. 10-2022-0148907 filed on Nov. 9, 2022 and Korean Patent Application No. 10-2023-0006560 filed on Jan. 17, 2023 in the Korean Intellectual Property Office, the disclosure of which is incorporated herein by reference.

### BACKGROUND

### 1. Field of the Invention

The following example embodiments relate to a system and method for supporting automated self-experimentation based on lifelog data for health behavior change. More particularly, the example embodiments relate to an automated system and method for supporting planning and conducting of self-experimentation for health behavior change based on lifelog data of a user collected through a smart device.

### 2. Description of the Related Art

Self-experimentation refers to one of special research methods in which an experimenter conducts an experiment on the experimenter himself or herself and is a method that is widely used in research related to psychology, education, and human behavior.

The self-experimentation is also well known as an N-of-1 trial research in which one experiment subject (generally, experimenter) randomly repeats and experiences treatment for an experiment group and a control group and verifies the effect for target outcome of the corresponding treatment.

The self-experimentation is considered as being particularly important in personalized medicine and patient-centered research and may be used in a process of exploring a personalized intervention and treatment method in the field of health behavioral change [1].

In particular, in terms of quantified self, users are collecting data related to their own states, behaviors, and surrounding environments from various smart devices in their daily life and are interested in a process of exploring and extracting new insight from the collected data.

Users that perform quantified self-desire to know what kind of relationship is present between different variables in addition to the overall trend of change over time or specific values from data in relation to their own health condition or behaviors [2].

Also, the users desire to verify factors (e.g., behavior, surrounding environment, etc.) that affect their health condition by directly conducting self-experimentation based on the collected data [3].

However, for most users that do not have much knowledge about data, statistics, and experimental design, a process of establishing and conducting self-experimentation may be slightly difficult.

Although a variety of data is collected from a smart device, a process of converting the data to major feature values related to a corresponding user's state, behavior, and surrounding environment is complex. Therefore, the user needs to rely on limited feature values provided from a manufacturer and has some difficulties in knowing which feature value needs to be further extracted.

A kind of hypothesis (e.g., smartphone usage before sleep is the cause of lowering my sleep quality) needs to be established before conducting self-experimentation. In the existing system, since a user may perform an approximate estimation from a correlation between variables through a graph on a dashboard, the user may simply conduct self-experimentation by randomly selecting one of various variables without clear grounds.

In particular, in the case of conducting self-experimentation related to health behavior, there is a situation in which a specific action needs to be performed or to not be performed and a system for guiding the situation is required. However, there is no such a system at present and thus, the user needs to perform an experiment while continuously remembering instructions for the corresponding action.

Non-patent documents are as follows:

0 [1] De Groot, Martijn, et al. “Single subject (N-of-1) research design, data processing, and personal science.” Methods of information in medicine 56.06 (2017): 416-418.
[2] Choe, Eun Kyoung, and Bongshin Lee. “Characterizing visualization insights from quantified selfers' personal data presentations.” IEEE computer graphics and applications 35.4 (2015): 28-37.
[3] Choe, Eun Kyoung, et al. “Understanding quantified-selfers' practices in collecting and exploring personal data.” Proceedings of the SIGCHI conference on human factors in computing systems. 2014.

### SUMMARY

Example embodiments provide an automated support system and method that may support even a user without much knowledge about data, statistics, and experiment design to plan and conduct self-experimentation based on data collected through a smart device of the user in daily life and to ultimately perform a health behavior change.

Example embodiments perform self-experimentation by extracting variables that represent a user's state, behavior, and surrounding environment through preprocessing of lifelog data collected through a smart device, by verifying a relationship between variables through causal inference, by receiving a recommendation on variables that require self-experimentation from a system, and by setting a detailed experimental scenario and environment.

According to an aspect, there is provided a system for supporting automated self-experimentation based on lifelog data for a health behavior change, the system including a data collection unit configured to collect data related to a user from a plurality of smart terminals and to preprocess the data as variables that represent a lifelog of the user; a causal inference unit configured to establish a causal relationship hypothesis between the variables of the user and to infer a causal relationship between the variables collected from the user using a statistical analysis method; a variable recommendation unit configured to recommend variables for verifying the causal relationship through self-experimentation for the user based on an inference result acquired from the causal inference unit and an interaction of the user with the system; and a self-experimentation planning unit configured to plan and conduct an experiment to verify the causal relationship through the self-experimentation for the user.

The data collection unit may include a data setting unit configured to represent a type of collectable and analyzable data according to a type of a smart terminal and to receive a selection from the user or to automatically select a type of data desired to collect; and a data preprocessing unit configured to collect data preselected by the data setting unit from the smart terminal, to preprocess the collected data, and to convert and verify the data to meaningful variables for analyzing the lifelog of the user that includes the user's state, behavior, or surrounding environment.

The causal inference unit may be configured to verify presence or absence of the causal relationship between the variables with respect to a plurality of variables that represents the lifelog of the user, and to, in response to a selection on a variable to be verified, provide information on variables required for analyzing the causal relationship between the variables and to provide a distribution change of confounding variables for classifying the causal relationship and correlation.

The variable recommendation unit may be configured to recommend variables based on an evaluation of the user, a category of interest of the user, and a user interaction on a causal inference unit usage history according to presence or absence of the causal relationship inferred by the causal inference unit, to predict a preference of the user based on the causal inference and the user interaction, to determine a series of priorities for recommended variables, and to suggest self-experimentation according to the priorities.

The self-experimentation planning unit may include an experimental scenario setting unit configured to set treatment and outcome variables that the user desires to verify and to verify or change a type of data collected from self-experimentation according to the set variables; and an experimental environment setting unit configured to set a method of providing intervention to be performed by the user by randomizing variables corresponding to an experiment period and a cause in a self-experimentation process.

According to another aspect, there is provided a method of supporting automated self-experimentation based on lifelog data for a health behavior change, the method including collecting, by a data collection unit, data related to a user from a plurality of smart terminals and preprocessing the data as variables that represent a lifelog of the user; establishing, by a causal inference unit, a causal relationship hypothesis between the variables of the user and inferring a causal relationship between the variables collected from the user using a statistical analysis method; recommending, by a variable recommendation unit, variables for verifying the causal relationship through self-experimentation for the user based on an inference result acquired from the causal inference unit and an interaction of the user with the system; and planning, by a self-experimentation planning unit, and conducting an experiment to verify the causal relationship through the self-experimentation for the user.

According to some example embodiments, through an automated support system and method, even a user without much knowledge about data, statistics, and experiment design may plan and conduct self-experimentation based on data collected through a smart device of the user in daily life and may ultimately perform a health behavior change.

Also, according to some example embodiments, it is possible to perform self-experimentation by extracting variables that represent a user's state, behavior, and surrounding environment through preprocessing of lifelog data collected through a smart device, by verifying a relationship between variables through causal inference, by receiving a recommendation on variables that require self-experimentation from a system, and by setting a detailed experimental scenario and environment.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 illustrates an overall operation process of a system for supporting automated self-experimentation based on lifelog data for a health behavior change according to an example embodiment;

FIG. 2 is a flowchart illustrating an example of a method of supporting automated self-experimentation based on lifelog data for a health behavior change according to an example embodiment;

FIG. 3 is a diagram illustrating a configuration of a system for supporting automated self-experimentation based on lifelog data for a health behavior change according to an example embodiment; and

FIG. 4 illustrates an example of describing variables required for analyzing a causal relationship according to an example embodiment.

### DETAILED DESCRIPTION

Hereinafter, some example embodiments will be described in detail with reference to the accompanying drawings. The following detailed structural or functional description of example embodiments is provided as an example only and various alterations and modifications may be made to the example embodiments. Accordingly, the example embodiments are not construed as being limited to the disclosure and should be understood to include all changes, equivalents, and replacements within the technical scope of the disclosure.

The terminology used herein is for describing various example embodiments only, and is not to be used to limit the disclosure. The singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Terms, such as first, second, and the like, may be used herein to describe components. Each of these terminologies is not used to define an essence, order or sequence of a corresponding component but used merely to distinguish the corresponding component from other component(s). For example, a first component may be referred to as a second component, and similarly the second component may also be referred to as the first component, without departing from the scope of the disclosure.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Regarding the reference numerals assigned to the elements in the drawings, it should be noted that the same elements will be designated by the same reference numerals, wherever possible, even though they are shown in different drawings. Also, in the description of embodiments, detailed description of well-known related structures or functions will be omitted when it is deemed that such description will cause ambiguous interpretation of the present disclosure.

As a larger number of smart terminals are used in daily life, types of data and an amount of data collected from such smart terminals are increasing. In this situation, it is difficult for an ordinary user to directly look at raw data and to plan self-experimentation from the raw data. In the example embodiments, through a process of automatically extracting feature values from data and converting the feature values to variables, it is possible to examine the range of variables that allow the user to conduct self-experimentation and to establish a scenario of the self-experimentation based on the range of variables.

According to the example embodiments, since the user may establish an experiment based on a causal relationship among the user's state, behavior, and surrounding environment that is analyzed by a system instead of randomly selecting variables and conducting the experiment, the user may perform efficient self-experimentation based on data. In particular, since the system proposed herein recommends variables that require self-experimentation based on a causal relationship result and a user reaction with the system, the user may quickly identify the necessary variables and may plan and conduct the experiment based on the identified variables.

The example embodiments provide an appropriate intervention while the user conducts self-experimentation and thus, may support the user to exactly perform a necessary action at a specific point in time during the experiment. Hereinafter, the example embodiments will be described with reference to the accompanying drawings.

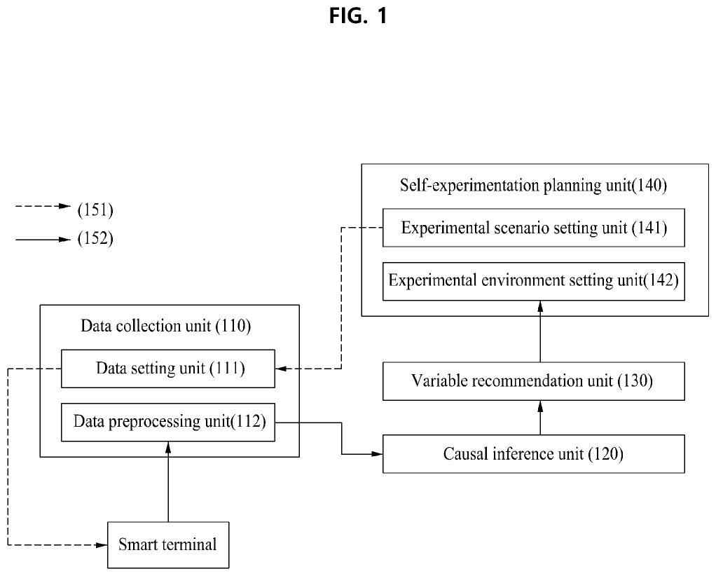

FIG. 1 illustrates an overall operation of a system for supporting automated self-experimentation based on lifelog data for a health behavior change (hereinafter, also referred to as an automated self-experimentation support system) according to an example embodiment.

The example embodiments are described based on an example in which a user with intermittent sleep disorder uses an interactive visual analysis system. This intermittent sleep disorder is provided as an example only and, without being limited thereto, the proposed automated self-experimentation support system may perform automated self-experimentation support based on lifelog data for a variety of the health behavior change.

According to example embodiments, a user may verify a cause that adversely affects the sleep quality of the user in daily life and may explore variables that require self-experimentation through recommendation from a system and then, may verify whether the corresponding cause actually adversely affects the sleep from establishment and execution of a self-experimentation plan.

Referring to FIG. 1, the automated self-experimentation support system according to an example embodiment includes a data collection unit 110, a causal inference unit 120, a variable recommendation unit 130, and a self-experimentation planning unit 140. In FIG. 1, a dotted line 151 represents a path of a data collection method and setting-related information and a solid line 152 represents a path of lifelog data and preprocessed variables.

The data collection unit 110 according to an example embodiment collects data related to a user from a plurality of smart terminals in daily life and preprocesses the data as variables that represent a lifelog of the user. The data collection unit 110 includes a data setting unit 111 and a data preprocessing unit 112.

The data setting unit 111 according to an example embodiment represents a type of collectable and analyzable data according to a type of a smart terminal and receives a selection from the user or automatically selects a type of data desired to collect.

The data preprocessing unit 112 according to an example embodiment collects data preselected by the data setting unit 111 from the smart terminal, preprocesses the collected data, and converts and verifies the data to meaningful variables for analyzing the lifelog of the user that includes the user's state, behavior, or surrounding environment.

The causal inference unit 120 according to an example embodiment supports a process of establishing a causal relationship hypothesis between the variables of the user and infers a causal relationship between the variables collected from the user using a statistical analysis method.

The variable recommendation unit 130 according to an example embodiment recommends variables between which the user needs to verify the causal relationship in a stricter manner through self-experimentation for the user based on a result acquired from the causal inference unit 120 and an interaction of the user with the system.

The self-experimentation planning unit 140 according to an example embodiment supports a process of planning and conducting an experiment such that the user may verify the causal relationship through the self-experimentation.

The self-experimentation planning unit 140 according to an example embodiment includes an experimental scenario setting unit 141 and an experimental environment setting unit 142.

The experimental scenario setting unit 141 according to an example embodiment guides a process of setting treatment and outcome variables that the user desires to verify and verifies or changes a type of data collected from self-experimentation according to the set variables.

The experimental environment setting unit 142 according to an example embodiment provides an intervention to be performed by the user by randomizing variables corresponding to an experiment period and a cause in a self-experimentation process.

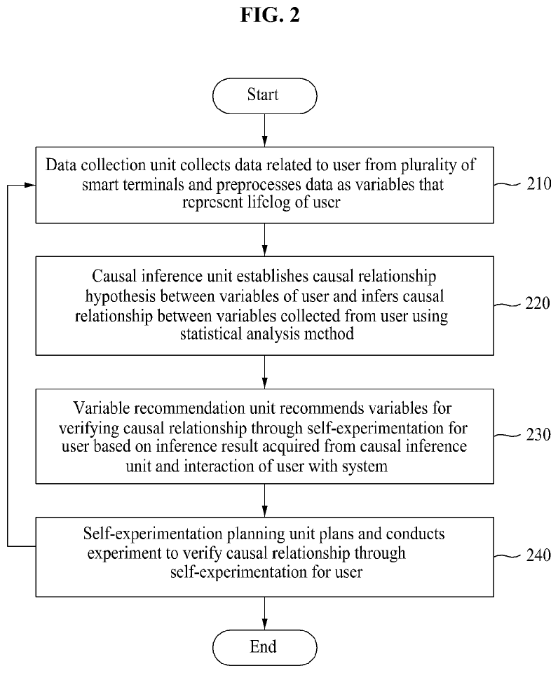

FIG. 2 is a flowchart illustrating an example of a method of supporting automated self-experimentation based on lifelog data for a health behavior change (hereinafter, also referred to as an automated self-experimentation support method) according to an example embodiment.

The proposed automated self-experimentation support method includes operation 210 of collecting, by a data collection unit, data related to a user from a plurality of smart terminals and preprocessing the data as variables that represent a lifelog of the user; operation 220 of establishing, by a causal inference unit, a causal relationship hypothesis between the variables of the user and inferring a causal relationship between the variables collected from the user using a statistical analysis method; operation 230 of recommending, by a variable recommendation unit, variables for verifying the causal relationship through self-experimentation for the user based on an inference result acquired from the causal inference unit and an interaction of the user with the system; and operation 240 of planning, by a self-experimentation planning unit, and conducting an experiment to verify the causal relationship through the self-experimentation for the user.

In operation 210, the data collection unit collects data related to the user from the plurality of smart terminals and preprocesses the data as variables that represent the lifelog of the user.

A data setting unit of the data collection unit represents a type of collectable and analyzable data according to a type of a smart terminal and receives a selection from the user or automatically selects a type of data desired to collect.

A data preprocessing unit of the data collection unit collects data preselected by the data setting unit from the smart terminal, preprocesses the collected data, and converts and verifies the data to meaningful variables for analyzing the lifelog of the user that includes the user's state, behavior, or surrounding environment.

In operation 220, the causal inference unit establishes the causal relationship hypothesis between the variables of the user and infers the causal relationship between the variables collected from the user using the statistical analysis method.

The causal inference unit verifies presence or absence of the causal relationship between the variables with respect to a plurality of variables that represents the lifelog of the user. In response to a selection on a variable to be verified, the causal inference unit provides information on variables required for analyzing the causal relationship between the variables and provide a distribution change of confounding variables for classifying the causal relationship and correlation.

In operation 230, the variable recommendation unit recommends variables for verifying the causal relationship through self-experimentation for the user based on an inference result acquired from the causal inference unit and an interaction of the user with the system.

The variable recommendation unit recommends variables based on an evaluation of the user, a category of interest of the user, and a user interaction on a causal inference unit usage history according to presence or absence of the causal relationship inferred by the causal inference unit. The variable recommendation unit predicts a preference of the user based on the causal inference and the user interaction, determines a series of priorities for recommended variables, and suggests self-experimentation according to the priorities.

In operation 240, the self-experimentation planning unit plans and conducts an experiment to verify the causal relationship through the self-experimentation for the user.

An experimental scenario setting unit of the self-experimentation planning unit sets treatment and outcome variables that the user desires to verify.

An experimental environment setting unit of the self-experimentation planning unit sets a method of providing intervention to be performed by the user by randomizing variables corresponding to an experiment period and a cause in a self-experimentation process.

The type of data to be collected and the intervention method determined through the experimental scenario setting unit and the experimental environment setting unit may be transmitted to the data collection unit and data may be collected according to the corresponding settings during the progress of the self-experimentation.

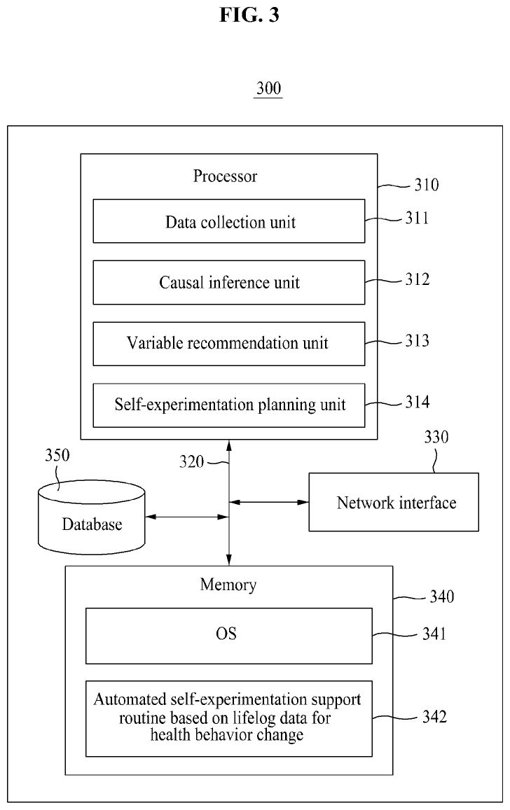

FIG. 3 is a diagram illustrating a configuration of a system for supporting automated self-experimentation based on lifelog data for a health behavior change according to an example embodiment.

Referring to FIG. 3, an automated self-experimentation support system 300 according to an example embodiment may include a processor 310, a bus 320, a network interface 330, a memory 340, and a database 350. The memory 340 may include an operating system (OS) 341 and an automated self-experimentation support routine based on lifelog data for health behavior change 342. The processor 310 may include a data collection unit 311, a causal inference unit 312, a variable recommendation unit 313, and a self-experimentation planning unit 314. According to other example embodiments, the automated self-experimentation support system 300 may include the number of components greater than or less than that of FIG. 3. However, there is no need to clearly illustrate many components according to the related art. For example, the automated self-experimentation support system 300 may include other components, such as a display and a transceiver.

The memory 340 may include a permanent mass storage device, such as random access memory (RAM), read only memory (ROM), and a disk drive, as non-transitory computer-readable recording media. A program code for the OS 314 and the automated self-experimentation support routine based on lifelog data for health behavior change 342 may be stored in the memory 340. Such software components may be loaded from another computer-readable recording media separate from the memory 340 using a drive mechanism (not shown). The other computer-readable recording media may include, for example, a floppy drive, a disk, a tape, a DVD/CD-ROM drive, and a memory card. According to other example embodiment, the software components may be loaded to the memory 340 through the network interface 330 instead of the computer-readable recording media.

The bus 320 enables communication and data transmission between components of the automated self-experimentation support system 300. The bus 320 may be configured using a high-speed serial bus, a parallel bus, a storage area network (SAN) and/or other appropriate communication technology.

The network interface 330 may be a computer hardware component for connecting the automated self-experimentation support system 300 to a computer network. The network interface 330 may connect the automated self-experimentation support system 300 to the computer network through wireless or wired connection.

The database 350 may serve to store and maintain all information required to support automated self-experimentation based on lifelog data for health behavior change. Although FIG. 3 illustrates that the database 350 is included in the automated self-experimentation support system 300, it is provided as an example only. The database 350 may be omitted according to a system implementation method or environment. Alternatively, all of or a part of the database 350 may be present as an external database constructed on a separate system.

The processor 310 may process an instruction of the computer program by performing basic arithmetic and logic operations and input/output operations of the automated self-experimentation support system 300. The instruction may be provided to the processor 310 through the bus 320 by the memory 340 or the network interface 330. The processor 310 may execute a program code for the data collection unit 311, the causal inference unit 312, the variable recommendation unit 313, and the self-experimentation planning unit 314. The program code may be stored in a recording device, such as the memory 340.

The data collection unit 311, the causal inference unit 312, the variable recommendation unit 313, and the self-experimentation planning unit 314 may be configured to perform operations 210 to 240 of FIG. 2.

The automated self-experimentation support system 300 may include the data collection unit 311, the causal inference unit 312, the variable recommendation unit 313, and the self-experimentation planning unit 314.

The example embodiments are described based on an example in which a user with intermittent sleep disorder uses an interactive visual analysis system. This intermittent sleep disorder is provided as an example only and, without being limited thereto, the proposed automated self-experimentation support system 300 may perform automated self-experimentation support based on lifelog data for a variety of health behavior change.

The data collection unit 311 according to an example embodiment collects data related to a user from a plurality of smart terminals and preprocesses the data as variables that represent a lifelog of the user.

The data collection unit 311 according to an example embodiment includes a data setting unit and a data preprocessing unit.

The data setting unit represents a type of collectable and analyzable data according to a type of a smart terminal and receives a selection from the user or automatically selects a type of data desired to collect.

The data preprocessing unit collects data preselected by the data setting unit from the smart terminal, preprocesses the collected data, and converts and verifies the data to meaningful variables for analyzing the lifelog of the user that includes the user's state, behavior, or surrounding environment.

The smart terminal according to an example embodiment includes, for example, a smartphone, a tablet, a wearable device, a smart speaker, an Internet of things (IoT) sensor, and collects the following mobile data related to sleep in daily life.

Lifelog data collectable from the smart terminal may include data automatically collected by the smart terminal and data manually input from the user through the smart terminal.

The data automatically collected by the smart terminal may include sensor data and interaction data occurring while the user is wearing or using the smart terminal.

A sensor within the smart terminal for collecting sensor data may include an inertial measurement unit (IMU), a photoplethysmogram (PPG) sensor, an electrocardiogram (ECG) sensor, a body temperature sensor, an impedance sensor, a carbon dioxide concentration sensor, a proximity sensor, an air pressure sensor, a temperature sensor, a global positioning system (GPS), a sensor for network connection state and data usage (wireless fidelity (Wi-Fi), long term evolution (LTE), 5G), a microphone, and a camera.

Smart terminal interaction data may include data related to screen ON/OFF, a type of screen interaction, an application use log, a call/conversation record, a short messaging service (SMS) usage history, and the like.

As data manually input from the user through the smart terminal, information that is difficult to automatically collect from the smart terminal or an item that requires subjective evaluation of the user may be collected in a form of a questionnaire or a self-report.

For example, default information of the user may include age, gender, occupation, and personality. User state information may include health (presence or absence of disease, fatigue, physical condition, etc.), a stress level, and an emotion. User activity information may include work (amount of work, difficulty level of work, etc.), meal (meal amount, meal composition, etc.), sleep (sleeping hours, perceived sleep quality, etc.), physical activity (type of exercise performed, exercise intensity, etc.), social activity, and hobbies.

User's relationship information may include a relationship between family members and a relationship between friends (presence of discord, period of discord, effect of discord on health condition and mental state of the user, etc.).

As types of analyzable variables, variables that represent the user's state, behavior, and surrounding environment related to sleep may be extracted by processing, such as converting and combining, the lifelog data.

Variables that represent the user's sleep state and environment may include sleeping hours, sleep state (heart rate during sleep, body temperature, non-rapid eye movement (REM) sleep), sleep activity (movement during sleep, snoring, number of awakenings, etc.), sleep environment (brightness, noise, air quality of a place of sleep), and the like.

Variables that may affect sleep in daily life may include an amount of physical activity (number of steps, amount of exercise), a work stress, an amount of work, previous sleep information (start time of sleep and amount of sleep), a health condition, a level of depression, an alcohol consumption, whether of eating, whether a smartphone is used before sleep, and the like.

The data setting unit according to an example embodiment may provide types of data that may be collected and variables that may be analyzed.

The data setting unit according to an example embodiment may use data that is automatically collected by the smart terminal.

As data that is automatically collected by the smart terminal, detailed information (e.g., data type, collection period, unit, example value) of sensor data that may be collected according to a manufacturer and a type of the smart terminal may be prestored in the system.

Types of variables related to the user's state, behavior, and surrounding environment that may be acquired through combination and conversion of corresponding sensor data are defined in the system. Therefore, types of available variables may be provided according to a smart terminal available by the user. For example, a variable, called a behavior of using a smartphone before sleep may be extracted by combining an app usage history, a time zone, and an ambient brightness.

Also, the system may preemptively recommend variables to the user to collect based on variables that are known as causes of sleep disorder in previous studies. The user may refer to the variables or may directly select a variable based on the existing knowledge about sleep of the user.

The data setting unit according to an example embodiment may use data that is manually input from the user through the smart terminal.

Data that is manually input from the user through the smart terminal may be supplemented through data that is manually input using variables that are difficult to extract from automatically collected data.

For example, among factors that may affect sleep, items that require subjective measurement of the user, such as work-related stress and a level of depression, need to be collected through a self-report method. Although this information may be inferred as sensor data, the self-report method may also be performed for higher accuracy.

The system may recommend additional data to be manually input from the user for analysis related to cause of sleep disorder based on the automatically collected data. The user may select data to actually use from among the data.

After selecting data to be collected, the user may verify variables finally available for analysis and the system may collect data from a plurality of smart terminals according to settings of the user.

The data preprocessing unit according to an example embodiment preprocesses data collected by the smart terminal according to settings by the data setting unit and converts the data to variables. A data preprocessing process may follow a general method employ for data analysis.

Initially, the data preprocessing unit performs processing of a missing value and an outlier. Here, the data preprocessing unit may remove the missing value or may replace the missing value with a representative value (e.g., average, median, mode). The data preprocessing unit may remove the outlier using an interquartile range method.

Then, the data preprocessing unit integrates data based on a specific time interval and extracts variables related to the user's state, behavior, or surrounding environment.

For example, GPS data may be classified into a plurality of places in which the user spends a lot of time through a clustering process and may be extracted as a variable called a meaningful place, such as home, work place, restaurant, and gym.

In the case of a domain that is repeated on a daily basis, such as sleep, variables may be generated by integrating data collected on a daily basis and by extracting a representative value (e.g., sum, average, standard deviation, count, maximum/minimum value, etc.) from the integrated data.

To establish a causal relationship, the order of an incident needs to be established. That is, a variable corresponding to cause needs to occur before effect. In an example embodiment, all the variables excluding an outcome variable that is sleep quality are extracted based on data collected before occurrence of sleep. For example, variables such as an amount of physical activity, a work stress, and an alcohol consumption, are extracted only based on events that occurred before occurrence of sleep on a corresponding day.

Also, variables of a previous day may also have an influence in addition to variables collected on the day and thus, may be extracted together. With the assumption that effect of a corresponding variable on sleep quality may decrease over time, a weight may be assigned. For example, assuming that a work stress level is weighted in inverse proportion to the past day, a corresponding variable may be extracted in a form of multiplying a stress level of n days ago by a value of 1/n.

A data sample is generated by preprocessing data in this manner and by collecting a plurality of variables in a single time interval.

A single data sample includes variable values related to the user's state, behavior, or surrounding environment collected in relation to sleep quality in a specific time interval and is used as a basic unit in causal inference analysis.

Preprocessed data is expressed in various forms, such as a picture, a graph, a text, and the like, and is extracted through sorting and filtering, such that the user may verify and understand a status of the user. For example, the user may verify various variables related to the user's state, behavior, or surrounding environment of the user over time as well as sleep quality.

The causal inference unit 312 according to an example embodiment establishes a causal relationship hypothesis between the variables of the user and infers a causal relationship between the variables collected from the user using a statistical analysis method.

The causal inference unit 312 according to an example embodiment verifies presence or absence of the causal relationship between the variables with respect to a plurality of variables that represents the lifelog of the user, and, in response to a selection on a variable to be verified, provides information on variables required for analyzing the causal relationship between the variables, and provides a distribution change of confounding variables for classifying the causal relationship and correlation.

The causal inference unit supports a process of establishes the causal relationship hypothesis between the variables of the user and inferring the causal relationship between the variables collected from the user using the statistical analysis method.

The causal inference unit 312 according to an example embodiment basically assumes a situation of determining whether the user's smartphone usage before sleep causes degradation in sleep quality.

When the user selects a variable estimated as the cause that degrades the sleep quality, the causal inference unit 312 according to an example embodiment provides presence or absence of the causal relationship and related detailed information.

The user may verify whether a causal relationship with sleep quality is present with respect to various variables. Variables to be explored may be recommended by the system based on correlation or may be directly selected and referred to by the user.

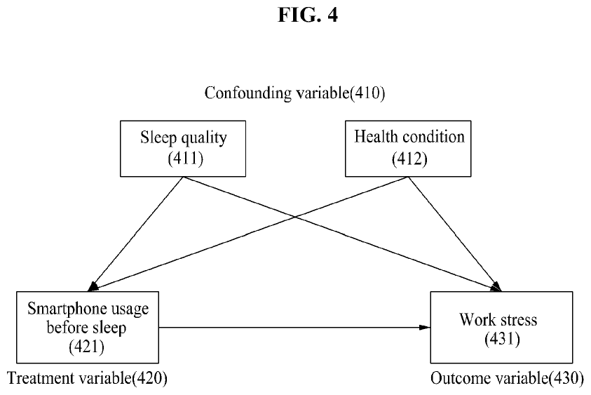

FIG. 4 illustrates an example of describing variables required for analyzing a causal relationship according to an example embodiment.

When a user selects a variable (i.e., potentially sleep quality 431) that the user desires to verify, a system provides information on a variable to be considered together in causal relationship analysis. For example, the system provides a confounding variable 410 that may affect the sleep quality 431 or all of the sleep quality 431 and a potential cause and thereby provides a variable to be controlled when the user performs a causal relationship verification.

Here, there may be used a graph in which each variable is expressed as a node and a relationship between variables is expressed as an edge. Here, magnitude and sign of correlation between variables may be expressed by changing a thickness and a color of the edge. For example, when whether a smartphone usage before sleep 421 that is a treatment variable 420 has a causal relationship with the sleep quality 431 that is an outcome variable 430 is to be explored, there may be a third confounding variable 410 (e.g., a work stress 411 and a health condition 412) that simultaneously affects two variables. In this case, such variables may be displayed in the graph such that the user may identify types of variables to be included and controlled in analysis.

The causal inference unit 312 according to an example embodiment may provide a change in distribution of confounding variables and a difference in sleep quality such that the user may understand a difference between a causal relationship and a correlation.

By providing the distribution of work stress levels on days with low smartphone usage and days with high smartphone usage, the user may verify how confounding variables are controlled in causal relationship analysis. Also, by finally providing the difference in the sleep quality according to the smartphone usage before sleep, the user may verify a causal relationship result according to control of confounding variables. Here, to help the user's understanding, text and the like may be used to provide explanation of the result.

The causal inference unit 312 according to an example embodiment may use a potential outcome framework that is a representative causal inference method in a causal relationship analysis method and may generate two virtual groups showing similar distribution of confounding variables and differ only in an estimation cause value that adversely affects the sleep quality.

For example, referring again to FIG. 4, when the estimated treatment variable 420 is the smartphone usage before sleep 421 and the work stress 411 is the confounding variable 410, samples that have as much similar values as possible for the work stress 411 and differ only in the smartphone usage before sleep 421 are matched and two groups are generated using only the samples.

Here, the two groups represent a high smartphone usage case and a low smartphone usage case, respectively. The samples may be classified into two groups based on an average or median of smartphone usage.

Propensity score matching, Mahalanobis distance matching, coarsened exact matching, and the like, which are well known in previous studies, may be employed as matching methods. Two groups with controlled confounding variables may be generated by matching most similar samples through a machine learning method or an artificial neural network.

Accordingly, through a matching process, a situation, such as a randomized controlled trial, may be generated even in an observational study without random allocation of samples and a causal relationship between variables may be inferred from the situation.

The variable recommendation unit 313 according to an example embodiment recommends variables for verifying the causal relationship through self-experimentation for the user based on an inference result acquired from the causal inference unit 312 and an interaction of the user with the system.

The variable recommendation unit 313 according to an example embodiment recommends variables based on an evaluation of the user, a category of interest of the user, and a user interaction on a causal inference unit usage history according to presence or absence of the causal relationship inferred by the causal inference unit 312. The variable recommendation unit 313 according to an example embodiment predicts a preference of the user based on the causal inference and the user interaction, determines a series of priorities for extracted variables, and suggests self-experimentation according to the priorities.

In causal inference-based variable recommendation according to an example embodiment, the variable recommendation unit 313 may recommend variables that the user needs to additionally verify through an experiment based on a result acquired from the causal inference unit 312. Types of corresponding variables may be recommended according to the following classification.

A case in which the causal inference unit 312 determines that the causal relationship is present may include a case in which if the analysis result of the causal relationship is not clear, presence or absence of the causal relationship is in a borderline (e.g., a case in which a p-value is slightly greater than 0.05 that is a reference value in correlation analysis between a corresponding variable and sleep quality) or a case in which if the causal relationship is present, but the corresponding variable has an insignificant influence on the sleep quality (e.g., a case in which an absolute value of a correlation coefficient between the corresponding variable and the sleep quality has a very small value close to 0).

Also, a case in which the analysis result of the causal relationship varies according to a selected data collection period may include a case in which the causal relationship is present or absent when changing the data collection period that is a target of analysis (e.g., a case in which the causal relationship was present for a specific period, but the causal relationship is absent when the period is changed) or a case in which a direction of the causal relationship is changed (e.g., a case in which a positive causal relationship for sleep quality was for a specific period, but a negative causal relationship is present for another period).

A case in which the causal inference unit 312 determines that the causal relationship is absent may include a case in which if many confounding variables have a close correlation with a treatment variable, it is difficult to find a statistically significant causal relationship when all confounding variables are controlled in a causal inference process (i.e., a case in which complex interaction between variables is estimated to be present), and may also include a case in which the causal relationship is known to be present in previous studies, but the causal relationship is determined to be absent for a user.

In user interaction-based variable recommendation according to an example embodiment, the variable recommendation unit 313 may recommend variables using an evaluation of the user on the inferred causal relationship, a category of interest of the user, or a causal inference unit usage history.

For example, due to usual low physical activity, the user may presume that there is no causal relationship with sleep quality, but may determine that the causal relationship is present from a result inferred by the system based on data.

As described above, when there is a discrepancy between factors affecting sleep quality known by the user and a result explored based on data, or when the corresponding inferred causal relationship is to be further examined, the user may separately display the corresponding variable using the causal inference unit 312 and, based thereon, the system may recommend variables.

Also, by allowing the user to specify a variable category that the user mainly desires to examine, the system may recommend variables that require self-experimentation within the corresponding variable category. For example, when the user desires to examine factors that affect the sleep quality mainly based on variables related to food and drink, the system may recommend variables (e.g., alcohol consumption, late-night snacking, etc.) that require self-experimentation of the user within the corresponding category.

In addition, the system may classify a category in which the user is highly interested based on types of variables with which the user mainly experimented in previous self-experimentation and may mainly recommend variables within the corresponding category or variables within another category highly related to the corresponding category.

The causal inference unit 312 may extract variables in which the user is highly interested based on an amount of time or the number of searches used for the user to examine a specific variable and may recommend variables that require self-experimentation based thereon.

The variable recommendation unit 313 may suggest self-experimentation according to a series of priorities for variables extracted using various methods that belong to causal inference-based and user interaction-based variable recommendation methods. The priorities may be directly set by the user. Alternatively, the system may predict preference of the user and automatically set priorities using a machine learning method for self-experimentation record previously performed by the user.

In the case of automatically setting priorities and suggesting variables based on user preference, a variable recommendation may be optimized by determining whether self-experimentation is performed in such a manner that the user actually selects a corresponding variable. Alternatively, a recommendation method may be improved by collecting evaluation of the user on the recommended variables.

Accordingly, through the variable recommendation unit 313, the user may receive a recommendation on a candidate group of variables that require self-experimentation in association with the sleep quality and may establish an approximate plan for the self-experimentation.

The self-experimentation planning unit 314 according to an example embodiment plans and conducts an experiment to verify the causal relationship through the self-experimentation for the user.

The self-experimentation planning unit 314 according to an example embodiment includes an experimental scenario setting unit and an experimental environment setting unit.

The experimental scenario setting unit sets treatment and outcome variables that the user desires to verify and verifies or changes a type of data collected from self-experimentation according to the set variables.

The experimental environment setting unit sets a method of providing intervention to be performed by the user by randomizing variables corresponding to an experiment period and a cause in a self-experimentation process.

The self-experimentation planning unit 314 according to an example embodiment allows the user to directly verify the cause recommended by the variable recommendation unit 313 through the self-experimentation and to add a new external third variable and to verify whether the corresponding variable causes degradation in the sleep quality. The system supports a series of processes in which the user plans and conducts such self-experimentation.

In an example embodiment, a situation in which a user conducts self-experimentation that not consuming caffeine may become the cause of improving sleep quality is assumed as an example.

Here, although it is found that caffeine intake has a causal relationship with the sleep quality, the effect thereof is insignificant and an analysis result is not clear. Considering this, the variable recommendation unit 313 suggests that the user verifies caffeine intake through self-experimentation.

The user may set a scenario for the self-experimentation through the system and may set a method of providing intervention of the system if necessary in a self-experimentation process.

The self-experimentation basically uses a method such as N-of-1 trial. Through this, by randomly distributing order of variables corresponding to cause, a causal relationship between a corresponding variable and the sleep quality may be verified while automatically controlling variables that may affect cause and effect, such as confounding variables.

Such random distribution may be performed for variables related to “behavior” that the user may determine to perform or not perform. As in the example embodiment, the system randomly distributes a case of consuming caffeine or a case of not consuming caffeine and compares the sleep quality according to each situation.

In this process, the system automatically processes the random distribution based on a preset random probability and the user performs an N-of-1 trial experiment by changing a treatment variable according to a method set by the system.

In the method, the user sets the user's own scenario and based on a result acquired from the variable recommendation unit 313 and sets a specific experimental environment according to the scenario. However, if the user fails in planning self-experimentation, the system may automatically plan and start the self-experimentation.

Here, the system may automatically plan and conduct the self-experimentation based on variables corresponding to high preference or interest of the user. Also, the system may induce the user to plan and conduct the self-experimentation that is configured using variables not normally explored by the user and to discover a new causal relationship among the user's state, behavior, and surrounding environment.

When a type of data to be collected and an intervention method for performing a treatment variable are determined by the experimental scenario setting unit and the experimental environment setting unit of the self-experimentation planning unit 314, the self-experimentation may start based thereon and the data collection unit 311 may collect data according to corresponding settings.

When a self-experimentation period ends, a causal relationship visualization unit may verify whether a corresponding variable is an actual cause that affects the sleep quality and the user may change the user's health behavior based thereon.

The experimental scenario setting unit according to an example embodiment guides a process of setting treatment and outcome variables that the user desires to verify and verifies or changes a type of data collected through self-experimentation according to a selected variable.

The experimental scenario setting unit according to an example embodiment allows the user to select a variable to be verified through the self-experimentation.

Basically, the user may select a variable suggested by the variable recommendation unit 313 as cause and may perform the self-experimentation. For example, the user may select whether of caffeine intake as a treatment variable from among variables recommended to be verified through the self-experimentation by the variable recommendation unit 313.

Also, whether to automatically collect a corresponding variable using a sensor of a mobile device or whether to receive a manual input of the corresponding variable through self-report of the user may be selected. In the case of extracting the corresponding variable as automatically collected data, the system may preemptively recommend data to be collected and a related smart terminal.

For example, in the case of consuming caffeine, the user may make a separate record every time the user consumes a caffeinated beverage or may automatically collect a caffeine intake behavior using a device, such as a cup linked to the system.

The system according to an example embodiment may provide information on variables (e.g., confounding variables) that may be reviewed together in a causal relationship scenario set by the user. Corresponding information may be provided based on the causal relationship analyzed from previously collected data and may include variables that are verified as the cause of degrading or improving the sleep quality.

In the self-experimentation, all confounding variables may be controlled through a random distribution process of the variables corresponding to the cause. However, whether a method for the N-of-1 trial experiment is properly performed may be evaluated by collecting data corresponding to the confounding variable together.

The user may select, from among such variables, a variable (i.e., data to be collected together) to be extracted together in the self-experimentation process. In particular, if a confounding variable having a high correlation with the variable corresponding to the cause is present, the confounding variable may be collected together based on a result verified by the causal inference unit 312 and may be reviewed for subsequent analysis.

The experimental environment setting unit according to an example embodiment sets a method of providing intervention to be performed by the user by randomizing variables corresponding to a cause and an experiment period in the self-experimentation process.

The user may directly set a period of the self-experimentation using the experimental environment setting unit. Alternatively, when the system calculates a minimum number of samples required for causal inference, the user may set the experiment period based thereon. Also, the user sets a method of randomizing a variable corresponding to a cause and providing intervention.

For example, in the case of comparing the sleep quality between days when the user consumes caffeine and days when the user does not consume caffeine, the system may provide an intervention message to prevent the user from consuming caffein on specific days for the user that normally consumes caffeine.

The corresponding message serves to inform that the user is currently in self-experimentation and at the same time, inform what value a variable corresponding to the cause needs to have (i.e., whether the user may consume caffeine on a corresponding day).

The user may directly set a point in time at which the corresponding message is provided and contents of the message. During the progress of actual self-experimentation, the user behaves according to the intervention message. Also, if separate settings by the user are absent, the intervention message may be transmitted at an optimal point in time at which the user may well verify the intervention message, using previously collected variable data related to the user's state, behavior, and surrounding environment, and compliance of the experiment may be improved. Also, the user may set how long a value of a specific variable is to be maintained in the process of randomizing variables corresponding to the cause.

For example, basically, the user may conduct the experiment by randomizing days when the user consumes caffeine and days when the user does not consume caffeine on a daily basis. However, in the case of verifying a relationship between a consecutive intake period (or a consecutive no-intake period) and the sleep quality, randomization may be performed based on a longer period (e.g., 3 days and 1 week) as a single unit.

Also, if a period set as a basic randomization unit is extended, effect related to caffeine intake or caffeine no-intake may affect the sleep quality for a longer period. Therefore, the experiment period may be set in consideration of an effect wash-out period of the intervention method.

Types of data to be collected and the intervention method determined through the experimental scenario setting unit and the experimental environment setting unit may be transmitted to the data collection unit 311, and data may be collected according to corresponding settings during the progress of the self-experimentation.

The example embodiments allow the user to explore a causal relationship by performing self-experimentation using lifelog data of the user, such that the user may discover various causal relationships not revealed among the user's state, behavior, and surrounding environment, and may perform health behavior change based on the discovered causal relationships.

Also, the example embodiments support a process of enhancing understanding on the user's own state, behavior, and surrounding environment by allowing the user to examine a causal relationship between variables and to receive a recommendation on variables based on the causal relationship.

The example embodiments recommend variables to be verified by the user through self-experimentation based on an analyzed causal relationship and the user may perform the self-experimentation in daily life through the system proposed herein and may verify the causal relationship in N-of-1 trial.

Also, the example embodiments may enhance the user's understanding on variables used for self-experimentation by preprocessing data collected through a smartphone of the user and by converting the data to significant variables that represent lifelog of the user.

The example embodiments may efficiently perform a variable selection by verifying a causal relationship inferred from data before selecting variables and a scenario for self-experimentation and, since variables are selected according to recommendation from the system, may conduct an experiment in order of priority, starting with a variable that requires the self-experimentation.

The example embodiments may represent the user's state, behavior, and surrounding environment using more various variables by using a variety of data automatically collected through a smart terminal as well as self-report data.

The systems and/or apparatuses described herein may be implemented using hardware components, software components, and/or a combination thereof. For example, apparatuses and components described herein may be implemented using one or more general-purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. A processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that the processing device may include multiple processing elements and/or multiple types of processing elements. For example, the processing device may include multiple processors or a processor and a controller. In addition, different processing configurations are possible, such as parallel processors.

The software may include a computer program, a piece of code, an instruction, or some combinations thereof, for independently or collectively instructing or configuring the processing device to operate as desired. Software and/or data may be embodied permanently or temporarily in any type of machine, component, physical equipment, virtual equipment, computer storage medium or device, or in a propagated signal wave capable of providing instructions or data to or being interpreted by the processing device. The software also may be distributed over network coupled computer systems so that the software is stored and executed in a distributed fashion. In particular, the software and data may be stored by one or more computer readable storage mediums.

The methods according to the example embodiments may be recorded in non-transitory computer-readable media including program instructions to implement various operations embodied by a computer. Also, the media may include, alone or in combination with the program instructions, data files, data structures, and the like. Program instructions stored in the media may be those specially designed and constructed for the purposes, or they may be of the kind well-known and available to those having skill in the computer software arts. Examples of non-transitory computer-readable media include magnetic media such as hard disks, floppy disks, and magnetic tape; optical media such as CD ROM disks and DVD; magneto-optical media such as floptical disks; and hardware devices that are specially to store and perform program instructions, such as read-only memory (ROM), random access memory (RAM), flash memory, and the like. Examples of program instructions include both machine code, such as produced by a compiler, and files containing higher level code that may be executed by the computer using an interpreter.

While this disclosure includes specific example embodiments, it will be apparent to one of ordinary skill in the art that various alterations and modifications in form and details may be made in these example embodiments without departing from the spirit and scope of the claims and their equivalents. For example, suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A system for supporting automated self-experimentation, the system comprising:
a data collection unit configured to collect data related to a user from a plurality of smart terminals and to preprocess the data as variables that represent a lifelog of the user;
a causal inference unit configured to establish a causal relationship hypothesis between the variables of the user and to infer a causal relationship between the variables collected from the user using a statistical analysis method;
a variable recommendation unit configured to recommend variables for verifying the causal relationship through self-experimentation for the user based on an inference result acquired from the causal inference unit and an interaction of the user with the system; and
a self-experimentation planning unit configured to plan and conduct an experiment to verify the causal relationship through the self-experimentation for the user.

2. The system of claim 1, wherein the data collection unit comprises:
a data setting unit configured to represent a type of collectable and analyzable data according to a type of a smart terminal and to receive a selection from the user or to automatically select a type of data desired to collect; and
a data preprocessing unit configured to collect data preselected by the data setting unit from the smart terminal, to preprocess the collected data, and to convert and verify the data to meaningful variables for analyzing the lifelog of the user that includes the user's state, behavior, or surrounding environment.

3. The system of claim 1, wherein the causal inference unit is configured to verify presence or absence of the causal relationship between the variables with respect to a plurality of variables that represents the lifelog of the user, and to, in response to a selection on a variable to be verified, provide information on variables required for analyzing the causal relationship between the variables and to provide a distribution change of confounding variables for classifying the causal relationship and correlation.

4. The system of claim 1, wherein the variable recommendation unit is configured to recommend variables based on an evaluation of the user, a category of interest of the user, and a user interaction on a causal inference unit usage history according to presence or absence of the causal relationship inferred by the causal inference unit, to predict a preference of the user based on the causal inference and the user interaction, to determine a series of priorities for recommend variables, and to suggest self-experimentation according to the priorities.

5. The system of claim 1, wherein the self-experimentation planning unit comprises:
an experimental scenario setting unit configured to set treatment and outcome variables that the user desires to verify and to verify or change a type of data collected from self-experimentation according to the set variables; and
an experimental environment setting unit configured to set a method of providing intervention to be performed by the user by randomizing variables corresponding to an experiment period and a cause in a self-experimentation process.

6. A method of supporting automated self-experimentation, the method comprising:
collecting, by a data collection unit, data related to a user from a plurality of smart terminals and preprocessing the data as variables that represent a lifelog of the user;
establishing, by a causal inference unit, a causal relationship hypothesis between the variables of the user and inferring a causal relationship between the variables collected from the user using a statistical analysis method;
recommending, by a variable recommendation unit, variables for verifying the causal relationship through self-experimentation for the user based on an inference result acquired from the causal inference unit and an interaction of the user with the system; and
planning, by a self-experimentation planning unit, and conducting an experiment to verify the causal relationship through the self-experimentation for the user.

7. The method of claim 6, wherein the establishing, by the causal inference unit, the causal relationship hypothesis between the variables of the user and the inferring the causal relationship between the variables collected from the user using the statistical analysis method comprises verifying presence or absence of the causal relationship between the variables with respect to a plurality of variables that represents the lifelog of the user, and in response to a selection on a variable to be verified, providing information on variables required for analyzing the causal relationship between the variables and providing a distribution change of confounding variables for classifying the causal relationship and correlation.

8. The method of claim 6, wherein the recommending, by the variable recommendation unit, the variables for verifying the causal relationship through the self-experimentation for the user based on the inference result acquired from the causal inference unit and the interaction of the user with the system comprises recommending variables based on an evaluation of the user, a category of interest of the user, and a user interaction on a causal inference unit usage history according to presence or absence of the causal relationship inferred by the causal inference unit, predicting a preference of the user based on the causal inference and the user interaction, determining a series of priorities for recommended variables, and suggesting self-experimentation according to the priorities.

9. The method of claim 6, wherein the planning, by the self-experimentation planning unit, and the executing the experiment to verify the causal relationship through the self-experimentation for the user comprises setting treatment and outcome variables that the user desires to verify and verifying or changing a type of data collected from self-experimentation according to the set variables, and setting a method of providing intervention to be performed by the user by randomizing variables corresponding to an experiment period and a cause in a self-experimentation process.

10. A non-transitory computer-readable recording medium storing instructions that, when executed by a processor, cause the processor to perform an operation method of an automated self-experimentation support system, the method comprising:
collecting, by a data collection unit, data related to a user from a plurality of smart terminals and preprocessing the data as variables that represent a lifelog of the user;
establishing, by a causal inference unit, a causal relationship hypothesis between the variables of the user and inferring a causal relationship between the variables collected from the user using a statistical analysis method;
recommending, by a variable recommendation unit, variables for verifying the causal relationship through self-experimentation for the user based on an inference result acquired from the causal inference unit and an interaction of the user with the system; and
planning, by a self-experimentation planning unit, and conducting an experiment to verify the causal relationship through the self-experimentation for the user.
